SEQUENCE LISTING (4 sequences)

SEQ ID NO: 1 - protein, 12 aa, Unknown
  Description of Unknown anti-NEQ C-terminal peptide
NEQEQPLGWHLS

SEQ ID NO: 2 - protein, 6 aa, Artificial Sequence
  Description of Artificial Sequence Synthetic 6xHis tag
HHHHHH

SEQ ID NO: 3 - protein, 158 aa, Homo sapiens
TNTTPHVPAEGPEASRPPKLHPGLRAAPGQEPPEHMAELQRNEQEQPLGQWHLSKRDTGAALLAESRAEKNRLWGPLEVRRVGRSSKQLVDIPEGQLEARAGRGRGQWLQVLGGGFSYLDISLCVGLYSLSFMGLLAMYTYFQAKIRALKGHAGHPAA

SEQ ID NO: 4 - protein, 15 aa, Homo sapiens
TNTTPHVPAEGPELI

(19) United States
(12) Patent Application Publication
(10) Pub. No.: US 2021/0156860 A1
(43) Pub. Date: May 27, 2021

(54) SYSTEMS AND METHODS FOR RAPID DIAGNOSTIC FOR VARIOUS CANCERS

(71) Applicant: Sapphire Biotech, Inc., San Diego, CA (US)

(72) Inventors: Sergei SVAROVSKY, San Diego, CA (US); Alim SEIT-NEBI, San Diego, CA (US); Catalina VALENCIA, San Diego, CA (US)

(21) Appl. No.: 16/841,521

(22) Filed: Apr. 6, 2020

Related U.S. Application Data
(60) Provisional application No. 62/829,556, filed on Apr. 4, 2019.

(51) Int. Cl. G01N 33/573

(52) U.S. Cl. CPC G01N 33/573; G01N 2333/90212; C12Y 108/03002

(57) ABSTRACT

A method for setting a threshold for basal levels of QSOX1-L in urine comprising: Storing de-identified urine from 100 BC patient samples and from 100 patients with non-malignant conditions; serially diluting the patient samples with a blocking buffer in triplicate followed by incubation in ELISA plates coated with anti-QSOX-L capture Ab; after 1-hour incubation at 37 C, washing plates followed by addition of biotinylated anti-QSOX-L detection antibody; using Streptavidin-HRP to generate dose dependent signal; obtaining a standard curve for each plate using recombinant QSOX1-L protein spiked into urine that has been depleted of QSOX1-L using affinity chromatography column conjugated with anti-sera against 100aa peptide; calculating concentrations of QSOX1-L based on a standard curve for each plate; and calculating a mean concentrations, ±2 SD to establish a reference range for QSOX1-L levels in urine from patients and individuals without malignant disease.

### CROSS-REFERENCE TO RELATED APPLICATIONS

This application claims priority to U.S. Provisional Patent Application No. 62/829,556, filed Apr. 4, 2019, the contents of which are incorporated herein by reference in their entirety for all purposes.

### SEQUENCE LISTING

The sequence listing submitted herewith in the ASCII text file entitled “127607-001UT1_Sequence_Listing” created Jan. 29, 2021, with a file size of 2,332 bytes, is incorporated herein by reference in its entirety.

### BACKGROUND

### 1. Technical Field

The embodiments described herein are related to cancer prevention, diagnosis, and treatment technologies to improve cancer outcomes in low- and middle-income countries, and low resource settings.

### 2. Related Art

Cancer remains one of the leading causes of morbidity and mortality worldwide. According to the WHO, cancer burden had risen to 18.1 million new cases and 9.6 million cancer deaths in 2018.2 While it's well known that cancer is a leading cause of death and disability worldwide, what is less recognized is the significant growth of cancer in the developing world. Only two decades ago, the percentage of new cases was similar for developed and developing regions. Today, 55 percent of new cases arise in developing nations—a figure that could reach 60 percent by 2020 and 70 percent by 2050.4 These disparities in cancer risk combined with poor access to epidemiological data, research, treatment, and cancer control and prevention combine to result in significantly poorer survival rates in developing countries for a range of malignancies.

For example, Bladder Cancer (BC) ranks 13th in terms of number of deaths, with mortality rates decreasing particularly in the most developed countries. The exceptions are countries undergoing rapid economic transition, including in Central and South America, China, central, southern, and eastern European countries. The observed patterns and trends of BC incidence worldwide appear to reflect the prevalence of tobacco smoking. This contrasts with steady decrease in smoking rates in industrialized nations. Emerging evidence also suggests that environmental factors such as chlorinated water may account for large number of new BC cases. Infection with Schistosoma haematobium is a well-documented risk factor and an important cause of BC in developing world. Early detection and access to advanced diagnostic modalities and cancer therapies has led to declines in the incidence and mortality of BC in developed countries not seen in less developed communities.

The presenting feature of many new BC cases is hematuria, and diagnostic work up for hematuria includes cystoscopy and upper tract imaging to detect urinary tract malignancies. Diagnosis of BC has not evolved considerably over the past decades. Cytoscopy remains the gold standard for the detection and follow up of BC. Cystoscopy is highly sensitive in the detection of most bladder tumors with reported sensitivities of approximately 90%. It is however an expensive and invasive procedure that incites anxiety and causes discomfort in patients undergoing the test that often results in adverse effects such as infection, frequency of urination, dysuria, and visible hematuria. Lastly, high recurrence and the frequent need for follow-up impose a very high financial burden on patients and their families. Non-invasive urine cytology, although effective in detecting high-grade tumors (75% sensitivity), is severely limited in the diagnosis of low-grade malignancies (25% sensitivity). Therefore, developing cost-effective and non-invasive strategies for the detection of BC is of paramount importance particularly in the low resource settings where at risk patients would not follow up due to the high cost of cystoscopy.

Urinary biomarkers can be useful diagnostic tools in BC as urine-based diagnostics offer a non-invasive and cost-effective means for BC detection. Despite significant progress in discovering differentially expressed urinary protein markers, there are only a few FDA approved commercial rapid tests on the market today. All these tests however lack sensitivity and specificity required to qualify as a screening tool for BC detection. For example, Bladder Tumor Antigen (BTA Stat® test by Bion Diagnostic Sciences, Redmond, Wash.) has low specificity due to benign genitourinary conditions; also it delivers false positives in patients with hematuria. BladderChek® test marketed by Alere detects a specific nuclear matrix protein NMP22 with a sensitivity of 49%-65% and a specificity of 40%-90%. The high variability of NMP22 means that it is not ideal for rapid, easy detection of BC. Similar to BTA, NMP22 sensitivity is impacted by other non-cancerous conditions such as hematuria or inflammation. The FDA approved this test as an aid in the diagnosis of patients at risk or with symptoms of BC. Another test, UBC® Rapid Test, measures soluble fragments of cytokeratins 8 and 18 in urine. Assays based on cytokeratins detection are limited by relatively high false positive rates and limited ability to detect low grade tumors. Although all tests mentioned above outperform cytology, none of them have been widely adopted by urologists and thus, their application has not reduced the need for cystoscopy.

Clearly, finding new BC biomarkers that alone (or in a combination with other biomarkers) yield non-invasive test that performs as well or better than cystoscopy would be a very significant new development.

### SUMMARY

Three main innovations are described below: (a) Novel cancer biomarker. This is the first time that clinically relevant QSOX1-L splice variant have been identified as a biomarker of BC and possibly other cancers in serum; (b) Generation of novel antibodies that selectively detect only this splice variant and not others; and (c) use of this biomarker to develop a rapid and cost-effective diagnostic test for bladder and possibly other urologic cancers non-invasively from urine.

These and other features, aspects, and embodiments are described below in the section entitled “Detailed Description.”

### BRIEF DESCRIPTION OF THE DRAWINGS

Features, aspects, and embodiments are described in conjunction with the attached drawings, in which:

FIG. 1 illustrates a sample of results of screening of 12 normal samples and 41 bladder cancer sera by Western blot. The detection was done with anti-QSOX-L (anti-NEQ) rabbit polyclonal. The results show overall higher expression of QSOX1-L in bladder cancer serum. Note, only one specific band was observed in the WB with the anti-NEQ Ab.

FIG. 2A illustrates schematics of the Lateral Flow Assay to capture QSOX-L isoform. Anti-NEQ Ab is conjugated to a red fluorescent latex bead 200 nm in diameter. 2F1 Ab is biotinylated. The pair of antibodies form a sandwich with QSOX-L antigen which is captured via biotin tag by the Polystreptavidin test line.

FIG. 2B illustrates a post-run images of the strips after LF assay.

FIG. 2C illustrates a western blot data of the serum samples used to run the LF assay. Samples 1-4 are normal donor serum. Samples 5-11 are bladder cancer sera. Sample 12 is a blank diluent. Sample 10 was identified as a NED (No existing disease, i.e. a patient that was treated and no evidence of disease was found by cystoscopy).

FIG. 3 illustrates a western blot data on pooled samples using pan-QSOX1 reactive 3A10 mAb. 1—Pooled normal serum from San Diego Blood Bank, 2—Pooled serum from Innovative Research, Inc; 5—Pooled serum from Sigma Corp; 3,4,6—Different pooled sera of BC patients.

FIG. 4 illustrates a primary structure of C-terminus of QSOX1-S (SEQ ID NO: 4) and QSOX1-L (SE ID NO: 3) and selection of peptides for polyclonal antibody development. Transmembrane domain of QSOX1-L. Peptide used for the development of polyclonal anti-NEQ antibody from Dr. Lake.

FIG. 5 illustrates that Substantial equivalence of the Anti-Peptide1 antibody illustrated in FIG. 4 with anti-NEQ antibody, demonstrated by Western Blot of normal and bladder cancer sample. Samples 1-7 are normal serum, while samples 8-14 are bladder cancer serum.

FIG. 6 illustrate the dose response to recombinant QSOX1 of the sandwich assay illustrated in FIG. 2A.

FIG. 7 illustrates QSOX1 assay of FIG. 2A functionality in human serum.

FIG. 8 illustrates the results of 200 bladder cancer serum samples using sandwich LFA QSOX1 assay of FIG. 2A.

FIG. 9 is a diagram illustrating a QSOX1 Autoantibody Assay in accordance with one embodiment. In the autoantibody assay, a tracer bead is conjugated with QSOX1.

FIG. 10 illustrates the dose response to model autoantibody (2F1), which shows that the assay of FIG. 9 can detects model autoantibody up to 1 ng/mL.

FIG. 11 illustrates a competitive lateral flow assay for QSOX1-L peptide. In the QSOX1-L peptide assay of FIG. 10, a tracer bead is conjugated with antibody generated in rabbits against QSOX1-L peptide.

FIG. 12 illustrates the dose response to QSOX-L peptide for the Optimized competitive assay of FIG. 11, which shows that the assay is a robust assay with LOD<1 ng/mL.

FIG. 13 illustrates a comparison between the LF competitive peptide assay of FIG. 11 vs. western blot (WB) with randomly picked normal and cancer serum samples.

FIG. 14 is a diagram illustrating a Rab-a-NEQ/2F1 Ab assay for detecting only QSOX1-l.

FIG. 15 illustrates the matching pattern between the assay of FIG. 14 and western blot assays for QSOX1-L.

FIG. 16 illustrates the representation of Rab-a-NEQ/2F1 LF assay of FIG. 14 results with normal and cancer serum samples, and that the assay detects only QSOX1-L.

FIG. 17 illustrates the representation of 3A10/2F1 assay of FIG. 2A results with normal and cancer serum samples, and illustrates the assay detects both QSOX1-L and QSOX1-S.

### DETAILED DESCRIPTION

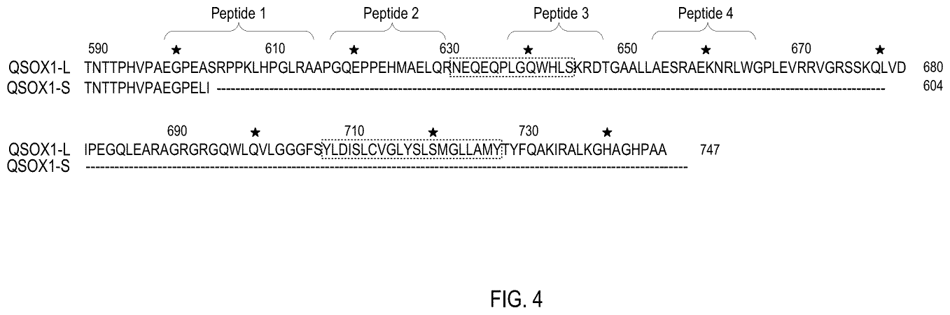

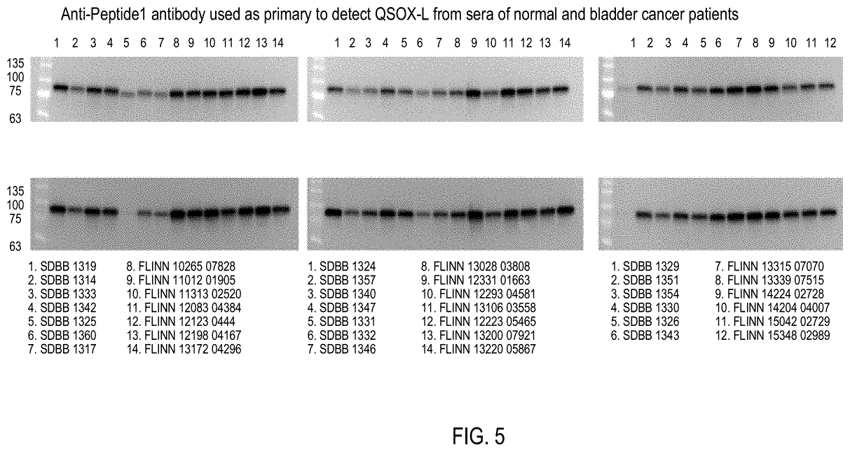

Preliminary Results: Quiescin sulfhydryl oxidase (QSCN6) is also called QSOX1. QSOX1 protein is composed of thioredoxin (Trx) and FAD-binding domains. Two splice variants are expressed: QSOX1-S, a short 604 amino acid secreted isoform, and QSOX1-L, a longer 747 amino acid isoform with a transmembrane domain (FIG. 4). QSOX1 facilitates disulfide bond formation during protein folding. There is ample evidence that QSOX1 is involved in tumorigenesis. In normal tissues QSOX1 is expressed at very low levels, but it is over-expressed in tumors. FIG. 5 illustrates that Substantial equivalence of the Anti-Peptide1 antibody illustrated in FIG. 4 with anti-NEQ antibody, demonstrated by Western Blot of normal and bladder cancer sample. Samples 1-7 are normal serum, while samples 8-14 are bladder cancer serum. As can be seen, there is overall higher QSOX1-L expression observed in cancer samples.

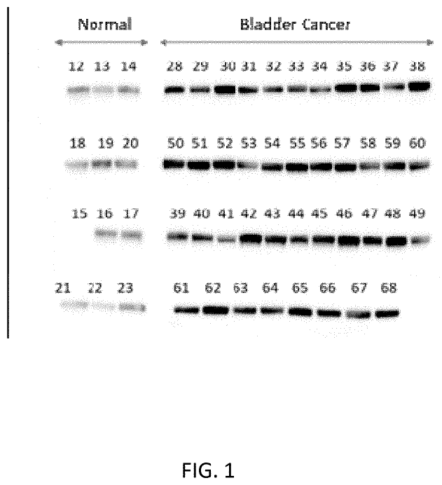

Differential Expression: Implementation of Rapid Diagnostic Test for QSOX1 in blood as a cancer biomarker are described herein. Using Western blot as an initial screening method it was determined that serum/plasma of bladder cancer patients had significantly elevated expression of QSOX1-L in ca. 200 patient samples. On the other hand, in normal donor samples the expression of QSOX-L was low (FIG. 1) and, in many cases, non-existent particularly in normal donor samples that were pooled (data not shown).

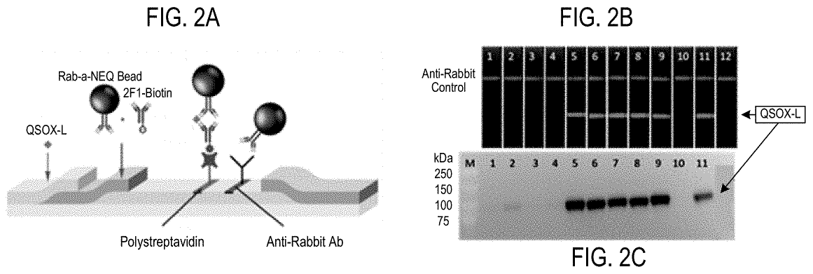

Lateral Flow Assays: During these preliminary studies, a series of sandwich Lateral Flow (LF) assays that measure either QSOX1-L or both QSOX1-L/S isoforms using monoclonal and polyclonal antibodies were developed and used as described herein. Mabs (3A10 and 2F1) recognize both QSOX1-S/L isoforms. A polyclonal antibody directed against a C-terminal peptide, NEQEQPLGWHLS (SEQ ID NO: 1), (hereafter referred to as anti-NEQ) recognizes only QSOX1-L isoform. Therefore, since the short isoform of QSOX1 (QSOX1-S) has been reported to be present in normal human serum, it can interfere with our detection of QSOX1-L because the biotinylated Mab used in the assays binds near the N-terminus of QSOX1. This interference problem provides a rationale for developing QSOX1-L-specific reagents as outlined in Aim #1 below. Nevertheless, the LF assays were validated by an independent Western Blot (WB) method and showed excellent correlation (FIG. 2). Having access to pairs of antibodies recognizing either QSOX1-S or QSOX1-L would be a great asset for increasing specificity of detection of QSOX1-S and -L biomarkers independently.

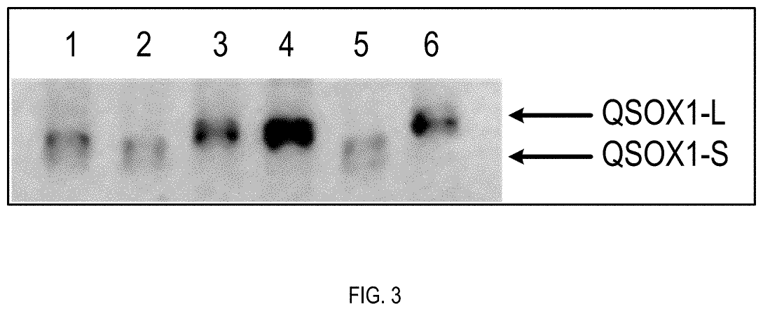

Moreover, a differential expression of QSOX1-L in cancer and QSOX1-S in non-cancer (FIG. 3) was observed. This presents a unique opportunity to take advantage of this expression pattern to increase statistical power of the assays. To do so we will have to develop antibodies that distinguish QSOX1-S and QSOX1-L. Since QSOX1-L isoform showed greatest differentiation between cancer and normal sera, it would be desirable first to develop a sandwich assay that detects only this isoform. Unfortunately, today there are no commercially available antibodies against QSOX1-L that do not cross-react with QSOX1-S. Therefore, our Aim #1 will be to generate a set of polyclonal antibodies (and eventually Mabs) intended to detect QSOX1-L in a sandwich assay with no cross-reactivity to QSOX1-S.

Aim #1: Development and validation of polyclonal antibodies that detect only QSOX1-L isoform.

Two different approaches were used to generate polyclonal QSOX1-L specific antibodies. In the first approach, rabbits are immunized with the entire recombinant 100aa C-terminal domain of QSOX1-L (FIG. 4) purified from 293F eukaryotic cells and conjugated to KLH. It is not known how the C-terminal domain of QSOX1-L is proteolytically cleaved, but presumably tumor-derived proteases are involved. Therefore, polyclonal antibodies to this entire domain are used to detect all possible proteolytic products of QSOX1-L and serve as a tool to deplete urine of QSOX1-L for true negative urine. In the second approach, five 12-15aa chemically synthesized KLH-conjugated peptides (derived from the 100aa C-terminal domain of QSOX1-L) are used as individual antigens (FIG. 4). The antibodies against short 12-15aa peptides are used in the assay development. Upon completion of immunization and evaluation of serum antibody titers by indirect ELISA, polyclonal antibodies are purified using corresponding peptide affinity columns.

Protein Expression and Purification: The 100aa C-terminal domain of QSOX1-L is synthesized de novo and cloned into pcDNA 3.1 with a natural QSOX1 signal peptide and a 6×His-tag (SEQ ID NO: 2) at the C-terminus. Integrity of protein expression vector is confirmed by DNA sequencing. The obtained vector is used to transfect 293F cells using Thermo's Freestyle system. Transfected cells is cultured for 7 days followed by harvest of supernatant and purification on a nickel affinity column. SDS-PAGE will confirm purity.

Peptide Synthesis and Conjugation: Five short peptides 12-15 (FIG. 4) derived from 100aa C-terminal domain of QSOX1-L have been designed and made at a qualified peptide synthesis facility (GL Biochem, USA) at 95% purity and 10 mg scale. Each peptide is conjugated to KLH for immunization and to bovine serum albumin (BSA) for serum titer evaluation and purified polyclonal antibodies tests, while unconjugated peptides are used for affinity column preparation. Recombinant 100aa C-terminal domain of QSOX1L and chemically synthesized peptides are conjugated to KLH and BSA via heterobifunctional SMCC linker using Pierce kits PN 77605 and 77115, respectively, following manufacturer's instructions.

Rabbits Immunization: For each antigen, two 6-8 week old healthy female New Zealand White rabbits were housed at a qualified animal facility (Abcore, Ramona, Calif.). Before primary immunization, 1-2 ml of pre-immune serum samples are collected as a control. For primary immunization, each rabbit will receive 100-200 μg dose of KLH-conjugated antigen emulsified with complete Freund's adjuvant (CFA) via subcutaneous injection. Subsequent immunizations were given every two weeks with antigen mixed in incomplete Freund's adjuvant (IFA) via the same route. Approximately 1-2 ml of serum will be collected from immunized animals after each boost one week after each injection for serum antibody titer evaluation by indirect ELISA with corresponding antigens. Serum antibody titers are expected to be at least 1:100,000 after a total of 4 injections. Once the titers are reached, rabbits will continue to receive immunization boosts and approximately 20 ml of serum form each animal will be collected 1 week after each injection for antibody purification.

Affinity Purification and Quality Control: Affinity column for anybody purification are prepared using Sulfo-link resin (Thermo Fisher PN 20401) reacted with cysteine-terminated peptides followed by serum purification per manufacturer's instructions. Purified antibodies were evaluated by indirect ELISA with corresponding peptide-BSA conjugate and recombinant QSOX1-L (received from D. Lake' lab) in the presence of human urine depleted of QSOX1-L. Urine samples depleted of QSOX1-L will be prepared from pooled urine samples collected from healthy donors using 100aa antibody affinity column. Antibodies will be validated in Western blot assay.

Milestone: Six polyclonal antibodies specific to QSOX1-L that bind native QSOX1-L in urine are validated and ready to use in Aim #2.

Aim #2: Establish quantitative sandwich ELISA and Lateral Flow Assay for QSOX-L from urine using antibodies from Aim #1.

Immunoblotting and immunoprecipitation: QSOX1-L was detected in urine using standard immunodetection techniques such as immunoblotting or immunoprecipitation. In immunoblotting, protein preparations from urine were electrophoresed through polyacrylamide gels and transferred onto nitrocellulose or PVDF membrane. The QSOX1-L was detected with polyclonal antibodies from Aim #1. In immunoprecipitation, a urine sample was incubated with antibody against QSOX1-L covalently coupled with agarose beads. Following washing, the bound QSOX1-L was detected by immunoblotting or ELISA.

Quantitative Sandwich ELISA Assay: Sandwich ELISA enables quantifying levels of proteins that allow the setting of a threshold for basal levels of QSOX1-L in urine. Stored de-identified urine from 100 BC patient samples were utilized in this study. Urine was also provided from 100 patients with non-malignant conditions. Urine was serially diluted with a blocking buffer in triplicate followed by incubation in ELISA plates coated with anti-QSOX-L capture Ab from Aim #1. After 1-hour incubation at 37 C, plates were washed followed by addition of biotinylated anti-QSOX-L detection antibody. Streptavidin-HRP was used to generate dose dependent signal. A standard curve was obtained for each plate using recombinant QSOX1-L protein spiked into urine that has been depleted of QSOX1-L using affinity chromatography column conjugated with anti-sera against 100aa peptide in Aim #1. Concentrations of QSOX1-L were calculated based on the standard curve for each plate. To establish a reference range for QSOX1-L levels in urine from patients and individuals without malignant disease, the mean concentrations, ±2 SD was calculated.

Quantitative Lateral Flow Assay (LFA): Lateral flow assays are essentially sandwich ELISA run on a nitrocellulose membrane. One antibody specific for QSOX1-L was conjugated onto detector beads. Another antibody, specific for another epitope of QSOX1-L was immobilized on a test line or biotinylated. If QSOX1-L is present in a sample, a sandwich complex will form, resulting in a signal on the test line when the detector beads accumulate at the test line as a result of direct sandwich formation or sandwich capture by the streptavidin zone.

Strip Design: The LFA was manufactured (American Bionostica, LLC) by affixing four overlapping pads to a 300 mm wide self-adhesive backing card. The card will be cut into 3 mm wide strips that are each inserted into a plastic cassette. The four different components of the assembly are: (1) a filter to remove particulates from urine; (2) the conjugate pad made of glass fiber, onto which assay reagents will be deposited and dried; (3) the nitrocellulose membrane that will contain two lines, a test line composed of either an anti-QSOX1-L antibody or a poly-streptavidin to capture biotinylated antibody, and a control line made of deposited anti-rabbit antibody to capture escaped beads functionalized with rabbit Ab. The control line ensures that the fluid flows properly through the membrane and the beads are released from conjugate pad; (4) absorbent pad that wicks away the moisture and promotes capillary flow on the nitrocellulose membrane.

LFA Configuration: Two configurations: (1) standard LFA configuration where one antibody is conjugated to detector beads and the other is dispensed as a test line; (2) alternative configuration where one antibody is conjugated to a detector bead and another is biotinylated while Polystreptavidin dispensed as a test line serves as a capture reagent for the sandwich formed by the two antibodies.

Reagent preparation: Each antibody from Aim #1 was conjugated to blue latex beads following a standard EDC/NHS conjugation chemistry and blocked with a proprietary blocking solution. Same antibodies will either be dispensed as a test line or biotinylated with Thermo EZ-link sulfo-NHS-LC2-Biotin (PN21343) per manufacturer's instructions. All possible working combinations of antibody pairs were tested to ensure optimal performance of the assay. The metrics of optimal performance will be highest dynamic range, lowest LOD and absence of non-specific binding.

Data Acquisition: During optimization the tests will be done in wet assay mode, where beads are dispensed in solution instead of being dried on a conjugate pad. The drying of the test and assembly into cassettes was relegated to Phase II. The aim is to generate standard curves with recombinant QSOX1-L in depleted urine and to determine limits of detection and dynamic range of each assay configuration. A universal reader RDS-1500 (Detekt Biomedical, Austin, Tex.) was used to quantify signals on the strips. The data was directly correlated to ELISA to ensure the two methods yield comparable results.

Milestones: (1) A correlation between sandwich ELISA and LF assays is established with at least one antibody pair; (2) Reproducibility, accuracy, limits of detection, and linear dynamic range of quantitative sandwich ELISA and LFA tests are determined. The tests are now available for screening samples in Aim #3.

In the QSOX protein assay of FIG. 2A, a tracer bead is conjugated with antibody 3A10. Another antibody 2F1 is biotin tagged. When serum containing QSOX protein is mixed with the reagents, a sandwich comprised of 3A10-Bead, QSOX and 2F1-biotin is formed. This complex is captured by polystreptavidin line to produce visible signal. The more QSOX is present, the more signal is observed. Control line consisting of capture anti-mouse antibody is added to ensure assay is working properly. The control line captures escaped 3A10-beads because 3A10 is a mouse antibody.

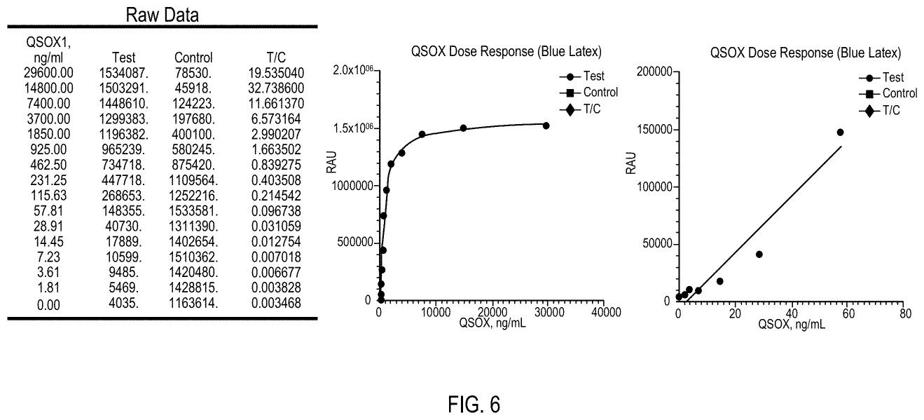

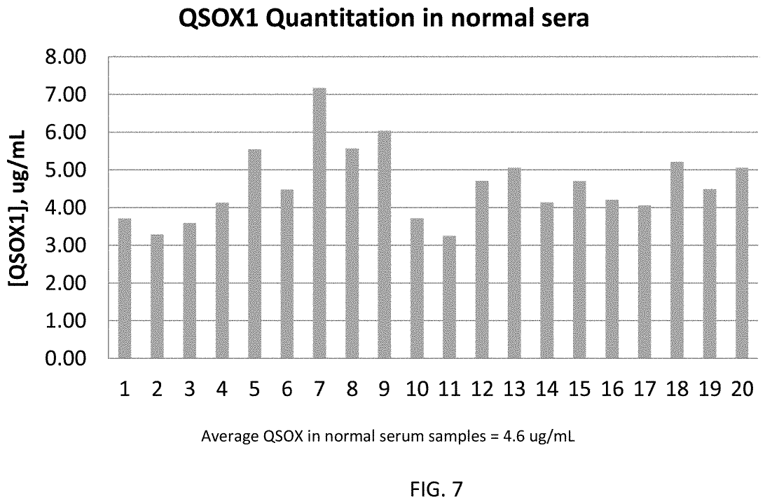

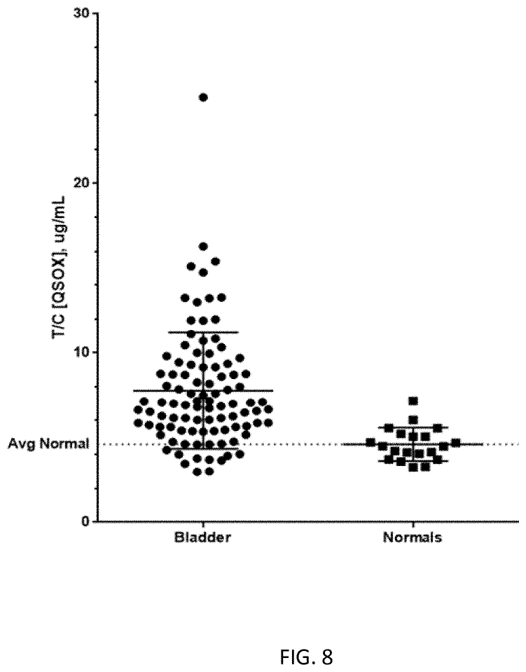

The data and graphs of FIG. 6 illustrate the dose response to recombinant QSOX1 of the sandwich assay illustrated in FIG. 2A. The results illustrate a very high 10,000× dynamic range; <20 ng/mL limit of detection. The graph of FIG. 7 illustrates QSOX1 assay functionality in human serum. The graph of FIG. 8 illustrates the results of 200 bladder cancer serum samples using sandwich LFA QSOX1 assay of FIG. 2A. As can be seen, although normal levels were in general found to be lower than cancer, the test could not differentiate different stages of bladder cancer.

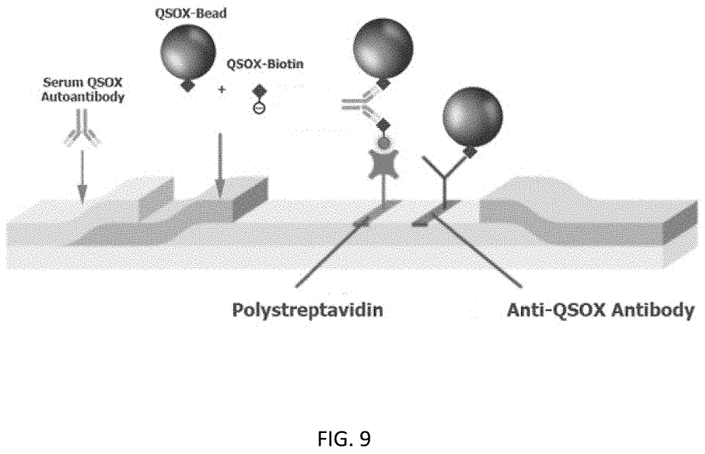

FIG. 9 is a diagram illustrating a QSOX1 Autoantibody Assay in accordance with one embodiment. In the autoantibody assay, a tracer bead is conjugated with QSOX1. Another QSOX1 is biotin tagged. When serum containing QSOX autoantibody is mixed with the reagents, a bridge is formed between autoantibody, a QSOX1 bead and QSOX1-biotin. This immunocomplex is captured by polystreptavidin line to produce visible signal. The more autoantibody is present, the more signal is observed. Control line consisting of capture anti-QSOX1 antibody is added to ensure assay is working properly.

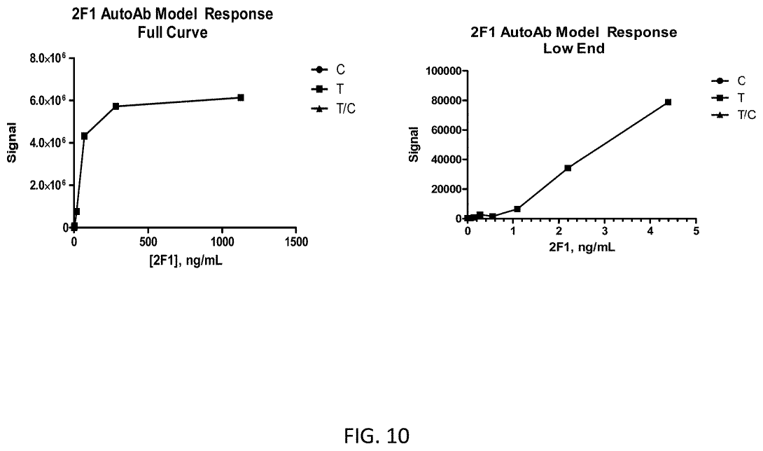

The graph of FIG. 10 illustrates the dose response to model autoantibody (2F1), which shows that the assay of FIG. 9 can detects model autoantibody up to 1 ng/mL.

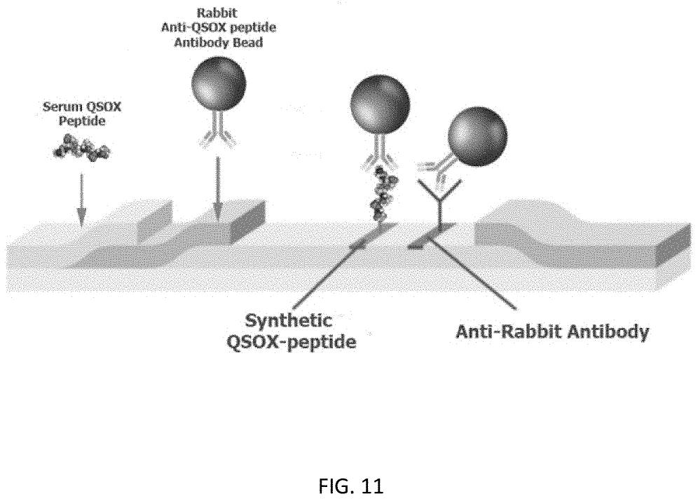

FIG. 11 illustrates a competitive lateral flow assay for QSOX1-L peptide. In the QSOX1-L peptide assay of FIG. 10, a tracer bead is conjugated with antibody generated in rabbits against QSOX1-L peptide. When serum containing QSOX1-L peptide is mixed with the reagent, the bead bound antibody reacts with peptide. A synthetic QSOX1 peptide is deposited on detection line. The synthetic peptide competes with natural peptide in serum for binding. The more peptide is present in serum the less binding occurs at the detection line. Hence the more peptide is in serum, the less signal is observed on the strip. This is called a competitive assay (frequently used for the detection of drugs of abuse). The Control line consisting of anti-rabbit antibody captures escaped beads to ensure assay is working properly.

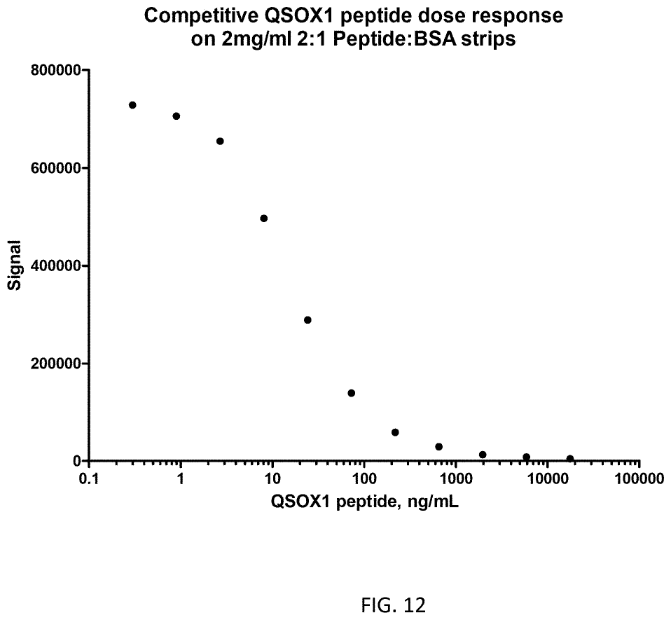

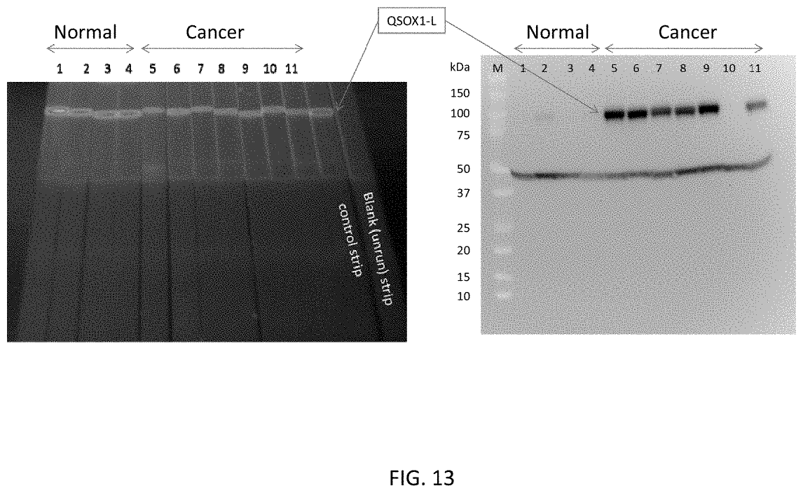

The graph of FIG. 12 illustrates the dose response to QSOX-L peptide for the Optimized competitive assay of FIG. 11, which shows that the assay is a robust assay with LOD<1 ng/mL. FIG. 13 illustrates a comparison between the LF competitive peptide assay of FIG. 11 vs. western blot (WB) with randomly picked normal and cancer serum samples. For the lateral flow strips, less signal means more QSOX1-L, while in the western blot more signal means more QSOX1-L.

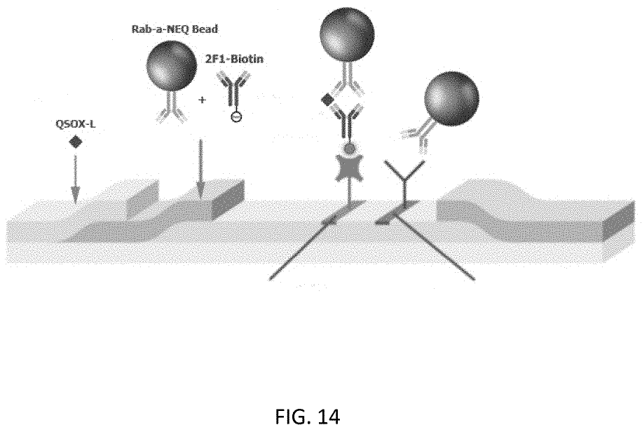

FIG. 14 is a diagram illustrating a Rab-a-NEQ/2F1 Ab assay for detecting only QSOX1-l. In the QSOX1-L protein assay of FIG. 14, a tracer bead is conjugated with antibody Rab-a-NEQ. Another antibody 2F1 is biotin tagged. When serum containing QSOX1-L protein is mixed with the reagents, a sandwich comprised of Rab-a-NEQ Bead, QSOX1-L and 2F1-biotin is formed. This complex is captured by polystreptavidin line producing visible signal. The more QSOX1-L is present, the more signal is observed. Control line consisting of capture anti-rabbit antibody is added to ensure assay is working properly. The control line captures escaped Rab-a-NEQ-beads because Rab-a-NEQ is a rabbit antibody.

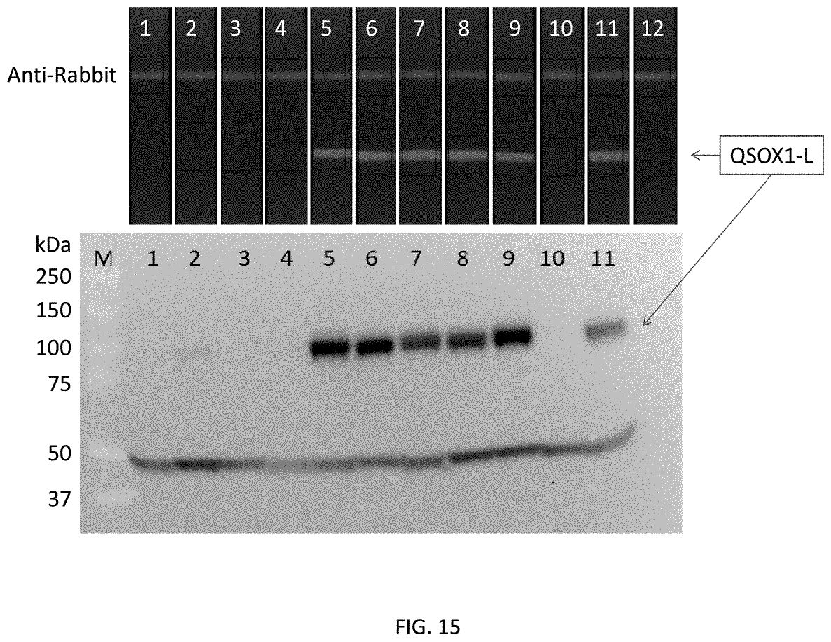

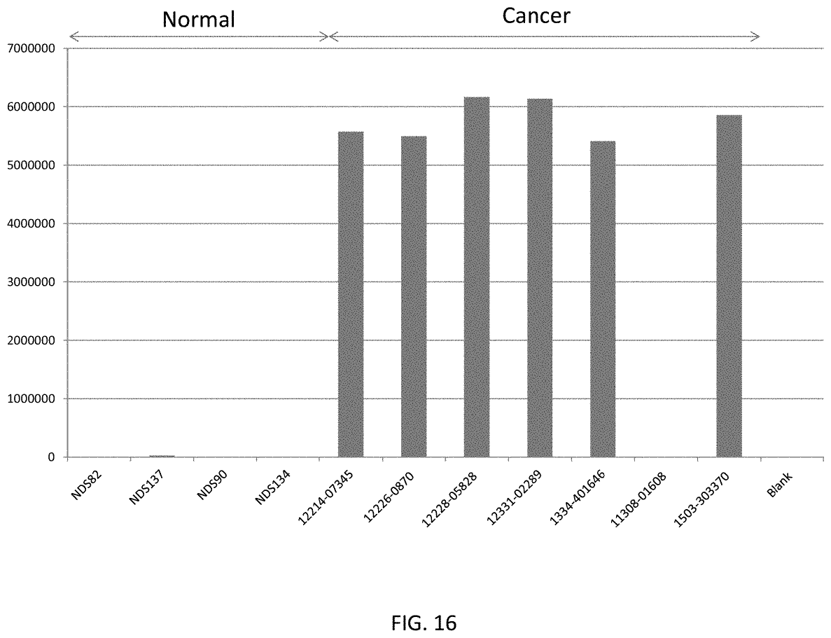

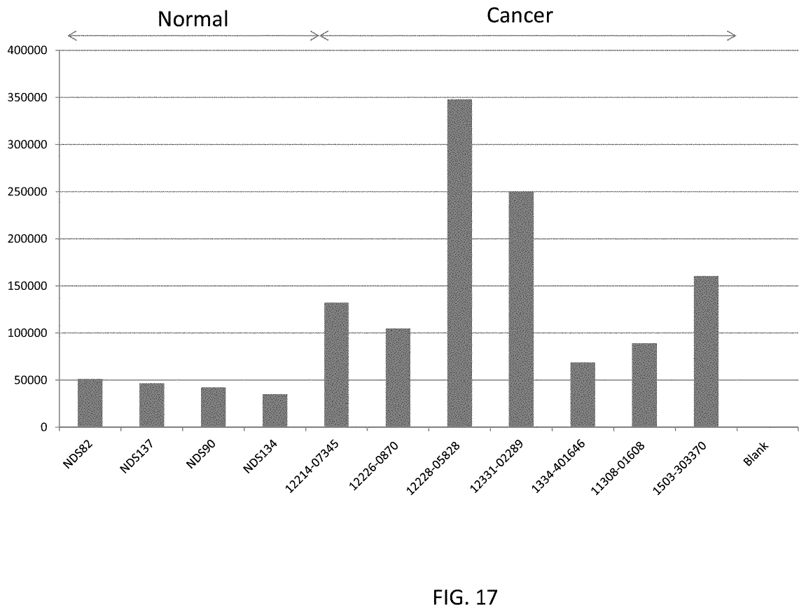

FIG. 15 illustrates the matching pattern between the assay of FIG. 14 and western blot assays for QSOX1-L. The bar graph of FIG. 16 illustrates the representation of Rab-a-NEQ/2F1 LF assay of FIG. 14 results with normal and cancer serum samples, and that the assay detects only QSOX1-L. Whereas the bar graph of FIG. 17 illustrates the representation of 3A10/2F1 assay of FIG. 2A results with normal and cancer serum samples, and illustrates the assay detects both QSOX1-L and QSOX1-S.

Aim #3: Screen 100 bladder cancer (BC) and 100 normal samples using LFA to estimate sensitivity and specificity of the assay.

Samples: The BC patient samples will consist of various stages including both muscle non-invasive (Ta/T1) and muscle invasive (T2/T3) BC urine samples. No evidence of disease (NED) samples were also included as controls. Normal samples were collected early morning (when concentration of biomarkers is highest) from age-matched group with no history of malignancy. De-identified urine from 100 consented BC patients were used in the study. Plasma was also provided from 100 patients with no history of malignancy who are age and gender-matched. QSOX1-L in urine was quantified by ELISA per protocol above and then compared to LFA results obtained with the same antibodies using similar capture/detector Ab configuration. A correlogram between ELISA and LFA was generated to confirm the results. In addition, a Western Blot assay was run on all samples to further confirm QSOX1-L identity. To establish a reference range for QSOX1-L levels in urine from patients and individuals without malignant disease, the mean concentrations, ±2 SD, was calculated.

Statistical Analysis: To determine positive and negative predictive values, an empirical receiving operating characteristic (ROC) curve was generated for QSOX1 ELISAs using a blind (re-coded) set of true positives and true negatives. ROC (0.20), the sensitivity of a QSOX1-based test with specificity 80%, will be estimated empirically, and the corresponding confidence interval was calculated based on normal approximation to the asymptotic distribution of a logit transformed ROC function. Area under the curve (AUC) and partial area under the curve (pAUC) on the interval [0, 0.2] was estimated for the ROC curve with respective confidence intervals. In LFA, for each test line and/or ratio of test/control, the ROC curve was calculated and plotted.

What is claimed:

1. A method for setting a threshold for basal levels of QSOX1-L in urine comprising:
storing de-identified urine from 100 BC patient samples and from 100 patients with non-malignant conditions;
serially diluting the patient samples with a blocking buffer in triplicate followed by incubation in ELISA plates coated with anti-QSOX-L capture Ab;
after 1-hour incubation at 37 C, washing plates followed by addition of biotinylated anti-QSOX-L detection antibody;
using Streptavidin-HRP to generate dose dependent signal;
obtaining a standard curve for each plate using recombinant QSOX1-L protein spiked into urine that has been depleted of QSOX1-L using affinity chromatography column conjugated with anti-sera against 100aa peptide;
calculating concentrations of QSOX1-L based on a standard curve for each plate; and
calculating a mean concentrations, ±2 SD to establish a reference range for QSOX1-L levels in urine from patients and individuals without malignant disease.
